(19) United States
(12) Patent Application Publication
(10) Pub. No.: US 2026/0335448 A1
(43) Pub. Date: Oct. 29, 2026

(54) CANNABINOID CONJUGATE MOLECULES COMPRISING AN AVERMECTIN COMPONENT

(71) Applicant: DIVERSE BIOTECH, INC., Miami, FL (US)

(72) Inventors: Paul HERSHBERGER, Miami, FL (US); Philip ARLEN, Miami, FL (US); William FISHER, Miami, FL (US)

(21) Appl. No.: 18/872,435

(22) PCT Filed: Jun. 6, 2023
(86) PCT No.: PCT/US2023/024537

§ 371 Date: Dec. 6, 2024

Related U.S. Application Data
(60) Provisional application No. 63/350,074, filed on Jun. 8, 2022.

(51) Int. Cl. A61K 47/55; A61K 47/54

(52) U.S. Cl. CPC A61K 47/55; A61K 47/549

(57) ABSTRACT

This disclosure provides multifunctional conjugate molecules in which at least one avermectin agent is covalently attached to at least one cannabinoid by means of a linker. The disclosed conjugate molecules are designed to deliver therapeutic benefits of components of the conjugate molecules.

Each reference cited in this disclosure is incorporated by reference herein in its entirety.

### TECHNICAL FIELD

This disclosure relates generally to multifunctional therapeutics.

### DETAILED DESCRIPTION

This disclosure describes multifunctional conjugate molecules comprising at least one avermectin component and at least one cannabinoid component covalently attached by a linker:

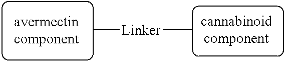

In contrast to traditional prodrugs, embodiments of the disclosed conjugate molecules are designed to deliver more than one therapeutic benefit via more than one mechanism of action; this is achieved when the covalent binding of the avermectin component to its target may accompany or facilitate the release of the cannabinoid at or near the site of the avermectin component's action, which can then effect a second therapeutic benefit. That is, these conjugate molecules are designed to deliver the therapeutic benefits of each of their components. In other embodiments, the avermectin component and the cannabinoid component are released to provide their respective therapeutic benefits via functionality of the linker.

### Conjugate Molecules

Conjugate molecules comprise at least one avermectin component covalently linked via a linker to at least one cannabinoid component.

In some embodiments, an avermectin component is covalently attached directly to a hydroxy or carboxylic acid group of a cannabinoid component. In some embodiments, cannabinoid conjugate components comprise an avermectin component and a cannabinoid component attached by means of a linker which is covalently attached at one end to the avermectin component and at the other end to a hydroxy or carboxylic acid group of the cannabinoid component. In some embodiments, the hydroxy group is an “aromatic hydroxy group;” i.e., a hydroxy group bonded directly to an aromatic hydrocarbon. In some embodiments, the hydroxy group is an “aliphatic hydroxy group;” i.e., a hydroxy group bound to a carbon that is not part of an aromatic ring.

In some embodiments, conjugate molecules contain only one avermectin component. In other embodiments, for example, when a cannabinoid component has at least two hydroxy groups, or at least one hydroxy group and at least one carboxylic acid group, or at least two carboxylic acid groups, conjugate molecules can contain two or more avermectin components, which can be the same or different.

In some embodiments, in which avermectin components are attached via a linker, the two or more linkers can be the same or different and, independently, the two or more avermectin components can be the same or different. Also independently, when a cannabinoid component contains two or more hydroxy groups, the two or more hydroxy groups can be aliphatic or the two or more hydroxy groups can be aromatic, or, for example, a first hydroxy group can be aliphatic and a second hydroxy group can be aromatic.

In some embodiments using particular types of linkers described below, conjugate molecules can contain two avermectin components which are both attached to a single linker. The two avermectin components can be the same or different.

In some embodiments, a conjugate molecule can contain an additional cannabinoid component.

Conjugate molecules can have one or more centers of asymmetry and can therefore be prepared either as a mixture of isomers (e.g., a racemic or diasteromeric mixture) or in an enantiomerically or diasteromerically pure form. Such forms include, but are not limited to, diastereomers, enantiomers, and atropisomers. Conjugate molecules can also include alkenes and can therefore be prepared either as a mixture of double bond isomers or independently as either an E or Z isomer. Isotopic variants of conjugate molecules can also be prepared.

Conjugate molecules can form salts. “Pharmaceutically acceptable salts” are those salts which retain at least some of the biological activity of the free (non-salt) compound and which can be administered as drugs or pharmaceuticals to an individual. Such salts, for example, include: (1) acid addition salts, formed with inorganic acids such as hydrochloric acid, hydrobromic acid, sulfuric acid, nitric acid, phosphoric acid, and the like; or formed with organic acids such as acetic acid, oxalic acid, propionic acid, succinic acid, maleic acid, tartaric acid and the like; (2) salts formed when an acidic proton present in the parent compound either is replaced by a metal ion, e.g., an alkali metal ion, an alkaline earth metal ion, or an aluminum ion; or coordinates with an organic base. Acceptable organic bases include ethanolamine, diethanolamine, triethanolamine and the like. Acceptable inorganic bases include aluminum hydroxide, calcium hydroxide, potassium hydroxide, sodium carbonate, sodium hydroxide, and the like. Further examples of pharmaceutically acceptable salts include those listed in Berge et al., Pharmaceutical Salts, J. Pharm. Sci. 1977 January; 66(1):1-19.

### Definitions

The following definitions apply to the descriptions of the “Avermectin Component(s)” and “Linkers” in the sections below and to the descriptions of “Group One Substituents” and “Group Two Substituents.”

“C1-C3 linear or branched alkyl” means “methyl, ethyl, propyl, and isopropyl.”

“C1-C8 linear or branched alkyl” means “methyl, ethyl, C3, C4, C5, C6, C7, and C8 linear alkyl and C3, C4, C5, C6, C7, and C8 branched alkyl.”

“C1-C3 linear or branched heteroalkyl” means “a linear or branched heteroalkyl containing 1, 2, or 3 carbon atoms.”

“C1-C8 linear or branched heteroalkyl” means “each of a C1, C2, C3, C4, C5, C6, C7, and C8 linear heteroalkyl and C1, C2, C3, C4, C5, C6, C7, and C8 branched heteroalkyl.”

“C1-C12 linear or branched heteroalkyl” means each of a C1, C2, C3, C4, C5, C6, C7, C8, C9, C10, C11, and C12 linear heteroalkyl and C1, C2, C3, C4, C5, C6, C7, C8, C9, C10, C11, and C12 branched heteroalkyl.”

“C1-C24 linear or branched heteroalkyl” means each of a C1, C2, C3, C4, C5, C6, C7, C8, C9, C10, C11, C12, C13, C14, C15, C16, C17, C18, C19, C20, C21, C22, C23, and C24 linear heteroalkyl and C1, C2, C3, C4, C5, C6, C7, C8, C9, C10, C11, C12, C13, C14, C15, C16, C17, C18, C19, C20, C21, C22, C23, and C24 branched heteroalkyl.”

“C1-C6 linear or branched alkoxyl” means “a linear or branched alkoxyl containing 1, 2, 3, 4, 5, or C carbon atoms.”

“C1-C6 linear or branched alkylamino” means “a linear or branched alkylamino containing 1, 2, 3, 4, 5, or 6 carbon atoms.”

“C1-C6 linear or branched dialkylamino” means “each linear or branched dialkylamino in which each alkyl independently contains 1, 2, 3, 4, 5, or 6 carbon atoms.”

“6-10-membered aromatic” means “each of a 6-, 7-, 8-, 9-, and 10-membered aromatic.”

“5- to 10-membered heteroaromatic” means “each of a 6-, 7-, 8-, 9-, and 10-membered heteroaromatic.”

“3- to 9-membered cycloheteroalkyl” means “each of a 3-, 4-, 5-, 6-, 7-, 8-, and 9-membered cycloheteroalkyl.

“C3-C6 cycloalkyl” means “C3, C4, C5, and C6 cycloalkyl.”

“Halide” means “Cl, Br, and I.”

“Group One Substituents” is a group of substituents consisting of:
(a) —OH;
(b) —NH₂;
(c) ═O;
(d) ═S;
(e) ═NR7, where R7 is H or is C1-C3 linear or branched alkyl or C1-C3 linear or branched heteroalkyl comprising an O, N, or S atom;
(f) —C(O)OR4, wherein R4 is H or C1-C3 linear or branched alkyl;
(g) —C(O)NR5R6, wherein R5 and R6 independently are H or C1-C6 linear or branched alkyl;
(h) halide;
(i) C1-C6 linear or branched alkoxyl;
(j) C1-C6 linear or branched alkylamino;
(k) C1-C6 linear or branched dialkylamino;
(l) 6- to 10-membered aromatic, optionally substituted with 1, 2, 3, or 4 substituents independently selected from
   (i) phenyl;
   (ii) halide;
   (iii) cyano;
   (iv) C1-C6 linear or branched alkyl, optionally substituted with
      (1) 1, 2, 3, 4, 5, 6, 7, 8, or 9 fluorine atoms; and/or
      (2) 1, 2, or 3 substituents independently selected from the Group Two Substituents; and
   (v) C1-C6 linear or branched heteroalkyl containing 1, 2, or 3 atoms independently selected from O, N, and S and optionally substituted with
      (1) 1, 2, 3, 4, 5, 6, 7, 8, or 9 fluorine atoms; and/or
      (2) 1, 2, or 3 substituents independently selected from the Group Two Substituents;
(m) 5- to 10-membered heteroaromatic, optionally substituted with 1, 2, 3, or 4 substituents independently selected from
   (i) phenyl;
   (ii) halide;
   (iii) cyano;
   (iv) C1-C6 linear or branched alkyl, optionally substituted with
      (1) 1, 2, 3, 4, 5, 6, 7, 8, or 9 fluorine atoms; and/or
      (2) 1, 2, or 3 substituents independently selected from the Group Two Substituents; and
   (v) C1-C6 linear or branched heteroalkyl containing 1, 2, or 3 atoms independently selected from O, N, and S and optionally substituted with
      (1) 1, 2, 3, 4, 5, 6, 7, 8, or 9 fluorine atoms; and/or
      (2) 1, 2, or 3 substituents independently selected from the Group Two Substituents;
(n) 3- to 9-membered cycloheteroalkyl having 1, 2, or 3 heteroatoms independently selected from O, N, and S, optionally substituted with 1, 2, 3, or 4 substituents independently selected from
   (i) phenyl;
   (ii) halide;
   (iii) cyano;
   (iv) C1-C6 linear or branched alkyl, optionally substituted with
      (1) 1, 2, 3, 4, 5, 6, 7, 8, or 9 fluorine atoms; and/or
      (2) 1, 2, or 3 substituents independently selected from the Group Two Substituents; and
   (v) C1-C6 linear or branched heteroalkyl containing 1, 2, or 3 atoms independently selected from O, N, and S and optionally substituted with
      (1) 1, 2, 3, 4, 5, 6, 7, 8, or 9 fluorine atoms; and/or
      (2) 1, 2, or 3 substituents independently selected from the Group Two Substituents; and
(o) C3-C6 cycloalkyl, optionally substituted with 1, 2, 3, or 4 substituents independently selected from
   (i) phenyl;
   (ii) halide;
   (iii) cyano;
   (iv) C1-C6 linear or branched alkyl, optionally substituted with
      (1) 1, 2, 3, 4, 5, 6, 7, 8, or 9 fluorine atoms; and/or
      (2) 1, 2, or 3 substituents independently selected from the Group Two Substituents; and
   (v) C1-C6 linear or branched heteroalkyl containing 1, 2, or 3 atoms independently selected from O, N, and S and optionally substituted with
      (1) 1, 2, 3, 4, 5, 6, 7, 8, or 9 fluorine atoms; and/or
      (2) 1, 2, or 3 substituents independently selected from the Group Two Substituents.

“Group Two Substituents” is a group of substituents consisting of:
(a) —OH;
(b) —NH₂;
(c) ═O;
(d) ═S;
(e) ═NR7, where R7 is H or is C1-C3 linear or branched alkyl or C1-C3 linear or branched heteroalkyl comprising an O, N, or S atom;
(f) —C(O)OR4, wherein R4 is H or C1-C3 linear or branched alkyl;
(g) —C(O)NR5R6, wherein R5 and R6 independently are H or C1-C6 linear or branched alkyl;
(h) halide;
(i) cyano;
(j) trifluoromethyl;
(k) C1-C6 linear or branched alkoxyl;
(l) C1-C6 linear or branched alkylamino;
(m) C1-C6 linear or branched dialkylamino;
(n) 6- to 10-membered aromatic; and
(o) 5- to 10-membered heteroaromatic comprising 1, 2, 3, 4, 5, or 6 heteroatoms independently selected from O, N, and S.

The definitions above apply to the descriptions that follow. For example, the phrase “R₄ is H or C1-C3 linear or branched alkyl” should be read as describing each of five sets of embodiments in which R₄ is H, R₄ is methyl, R₄ is ethyl, R₄ is propyl, and R₄ is isopropyl, respectively.

### Avermectin Component(s)

An “avermectin component” as used in this disclosure is that portion of an avermectin agent that is present in a conjugate molecule and covalently attached to a linker. A number of avermectin agents can be used to provide an avermectin component of a conjugate molecule. An “avermectin agent” is a member of the avermectin family or an active metabolite or derivative thereof. Examples of avermectin derivatives are described, for example, in Huang et al., Applied and Environmental Microbiology 81 (16), 5325-34, 2015; Batiha et al., Pharmaceuticals (Basel) 13 (8), 196, 2020.

In some embodiments, the avermectin component is provided by avermectin:

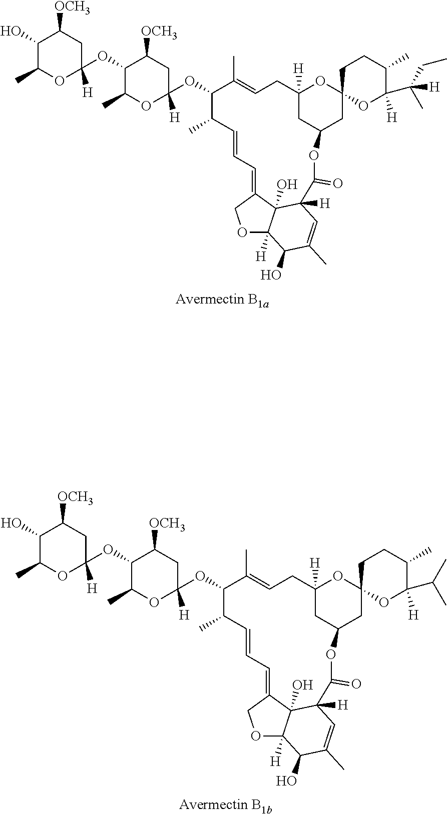

The avermectin can be avermectin B₁ₐ or avermectin B_{1b}.

In some embodiments, the avermectin component is provided by ivermectin:

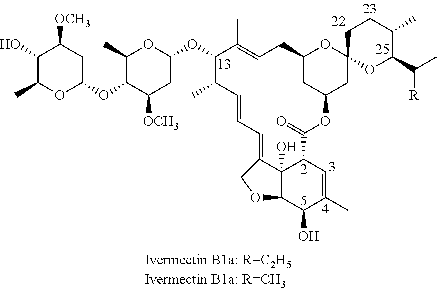

The ivermectin can be ivermectin B₁ₐ or ivermectin B_{1b}.

In some embodiments, the avermectin component is provided by emamectin:

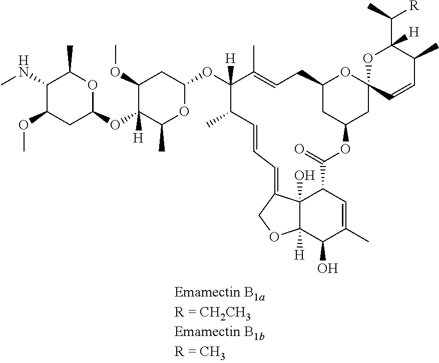

The emamectin can be emamectin B₁ₐ or emamectin B_{1b}.

In some embodiments, the avermectin component is provided by moxidectin:

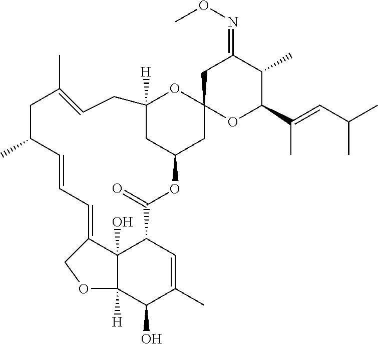 [SMILES: CO/N=C1\C[C@]2(C[C@@H]3C[C@@H](C/C=C(\C)C[C@@H](C)/C=C/C=C4\CO[C@@H]5[C@H](O)C(C)=C[C@@H](C(=O)O3)[C@]45O)O2)O[C@H](/C(C)=C/C(C)C)[C@H]1C]

In some embodiments, the avermectin component is provided by eprinomectin:

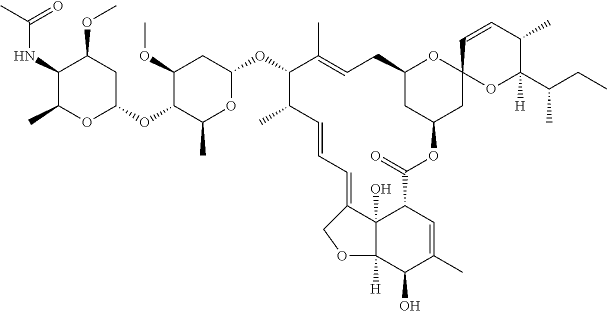 [SMILES: CC[C@H](C)[C@H]1O[C@]2(C=C[C@@H]1C)C[C@@H]1C[C@@H](C/C=C(\C)[C@@H](O[C@H]3C[C@H](OC)[C@@H](O[C@H]4C[C@H](OC)[C@H](NC(C)=O)[C@H](C)O4)[C@H](C)O3)[C@@H](C)/C=C/C=C3\CO[C@@H]4[C@H](O)C(C)=C[C@@H](C(=O)O1)[C@]34O)O2]

In some embodiments, the avermectin component is provided by doramectin:

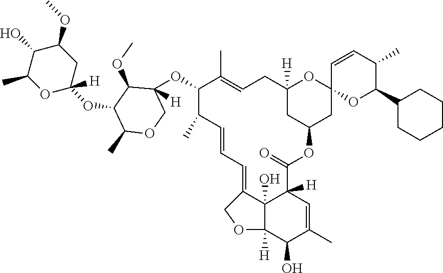

In some embodiments, the avermectin component is provided by selamectin:

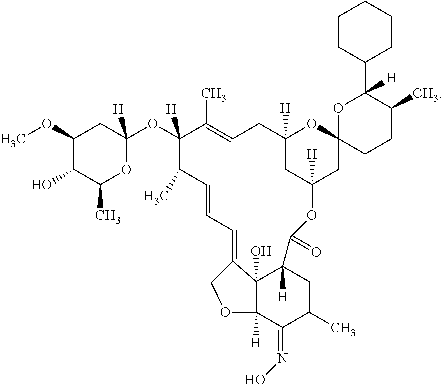

### Linkers

In some embodiments, linkers used to connect an avermectin component and a cannabinoid component are typically two to 10 atoms in length and are functionalized to facilitate release of the cannabinoid. In some embodiments, this release may occur approximately when the avermectin agent engages its biological target.

A variety of linkers can be used in the conjugate molecules. Examples are shown below.

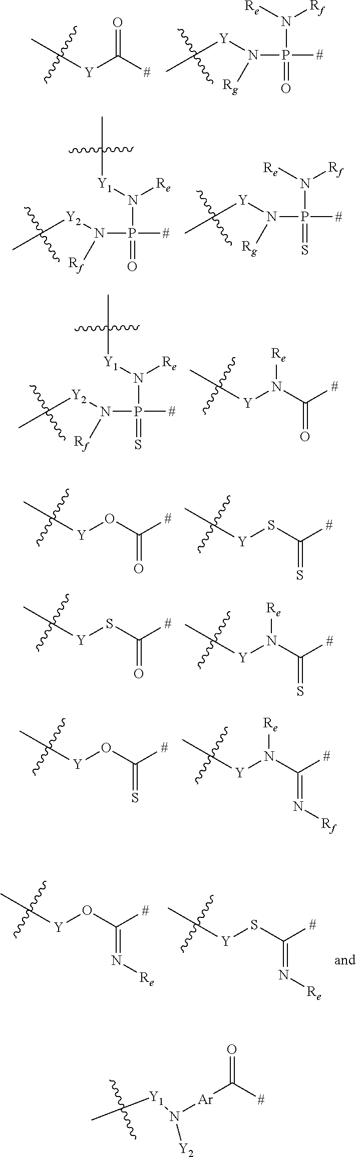

in which [symbol] marks a bond attaching the linker to the avermectin component, # indicates a site of covalent attachment to the cannabinoid component, and in which:
Y, Y₁, and Y₂ independently are absent or Y, Y₁, and Y₂ independently are selected from the group consisting of:
   (a) C1-C12 linear or branched alkyl, optionally substituted with
      (1) 1, 2, 3, 4, 5, 6, 7, 8, or 9 fluorine atoms; and/or
      (2) 1, 2, or 3 substituents selected from the Group One Substituents;
   (b) C2-C12 linear or branched alkenyl, optionally substituted with
      (1) 1, 2, 3, 4, 5, 6, 7, 8, or 9 fluorine atoms; and/or
      (2) 1, 2, or 3 substituents selected from the Group One Substituents;
   (c) C1-C12 linear or branched heteroalkyl containing 1, 2, 3, or 4 heteroatoms independently selected from O, N, and S, optionally substituted with
      (1) 1, 2, 3, 4, 5, 6, 7, 8, or 9 fluorine atoms; and/or
      (2) 1, 2, or 3 substituents selected from the Group One Substituents;
   (d) a 6- to 10-membered aromatic, optionally substituted with 1, 2, 3, or 4 substituents independently selected from the group consisting of:
      (1) phenyl,
      (2) halide,
      (3) C1-C6 linear or branched alkyl, optionally substituted with
         (i) 1, 2, 3, 4, 5, 6, 7, 8, or 9 fluorine atoms; and/or
         (ii) 1, 2, or 3 substituents independently selected from the Group Two Substituents, and
      (4) C1-C6 linear or branched heteroalkyl containing 1, 2, or 3 atoms independently selected from O, N, and S and optionally substituted with
         (i) 1, 2, 3, 4, 5, 6, 7, 8, or 9 fluorine atoms; and/or
         (ii) 1, 2, or 3 substituents independently selected from the Group Two Substituents;
   (e) a 6- to 10-membered heteroaromatic comprising 1, 2, 3, 4, 5, or 6 heteroatoms independently selected from O, N, and S and optionally substituted with 1, 2, 3, or 4 substituents independently selected from
      (1) phenyl,
      (2) halide,
      (3) trifluoromethyl,
      (4) C1-C6 linear or branched alkyl, optionally substituted with
         (i) 1, 2, 3, 4, 5, 6, 7, 8, or 9 fluorine atoms; and/or
         (ii) 1, 2, or 3 substituents independently selected from the Group Two Substituents, and
      (5) C1-C6 linear or branched heteroalkyl containing 1, 2, or 3 atoms independently selected from O, N, and S and optionally substituted with
         (i) 1, 2, 3, 4, 5, 6, 7, 8, or 9 fluorine atoms; and/or
         (ii) 1, 2, or 3 substituents independently selected from the Group Two Substituents; and
   (f) a C1-C24 linear or branched heteroalkyl containing 1, 2, 3, 4, 5, 6, 7, or 8 heteroatoms independently selected from O, N, and S, optionally substituted with
      (i) 1, 2, 3, 4, 5, 6, 7, 8, or 9 fluorine atoms; and/or
      (ii) 1, 2, 3, 4, 5, or 6 substituents selected from the Group One Substituents;
Ar is either:
   (a) a 6- to 10-membered aromatic, optionally substituted with 1, 2, 3, or 4 substituents independently selected from the group consisting of:
      (1) phenyl,
      (2) halide,
      (3) C1-C6 linear or branched alkyl, optionally substituted with
         (i) 1, 2, 3, 4, 5, 6, 7, 8, or 9 fluorine atoms; and/or
         (ii) 1, 2, or 3 substituents independently selected from the Group Two Substituents; or
   (b) a 6- to 10-membered heteroaromatic comprising 1, 2, 3, 4, 5, or 6 heteroatoms independently selected from O, N, and S and optionally substituted with 1, 2, 3, or 4 substituents independently selected from
      (1) phenyl,
      (2) halide,
      (3) trifluoromethyl,
      (4) C1-C6 linear or branched alkyl, optionally substituted with
         (i) 1, 2, 3, 4, 5, 6, 7, 8, or 9 fluorine atoms; and/or
         (ii) 1, 2, or 3 substituents independently selected from the Group Two Substituents, and
      (5) C1-C6 linear or branched heteroalkyl containing 1, 2, or 3 atoms independently selected from O, N, and S and optionally substituted with
         (i) 1, 2, 3, 4, 5, 6, 7, 8, or 9 fluorine atoms; and/or
         (ii) 1, 2, or 3 substituents independently selected from the Group Two Substituents; and
Rₑ, R_{f}, and R_{g} independently are selected from the group consisting of:
   (a) H;
   (b) C1-C8 linear or branched alkyl, optionally substituted with
      (1) 1, 2, 3, 4, 5, 6, 7, 8, or 9 fluorine atoms; and/or
      (2) 1, 2, or 3 substituents independently selected from the Group One Substituents;
   (c) C1-C8 linear or branched heteroalkyl containing 1, 2, or 3 heteroatoms independently selected from O, N, and S and optionally substituted with
      (1) 1, 2, 3, 4, 5, 6, 7, 8, or 9 fluorine atoms; and/or
      (2) 1, 2, or 3 substituents independently selected from the Group One Substituents;
   (d) phenyl, optionally substituted with 1, 2, or 3 substituents independently selected from the group consisting of:
      (1) C1-C6 linear or branched alkyl, optionally substituted with
         (i) 1, 2, 3, 4, 5, or 6 fluorine atoms; and/or
         (ii) 1 or 2 substituents independently selected from the Group Two Substituents; and
      (2) C1-C6 linear or branched heteroalkyl containing 1 or 2 heteroatoms independently selected from O, N, and S and optionally substituted with
         (i) 1, 2, 3, 4, 5, or 6 fluorine atoms; and/or
         (ii) 1 or 2 substituents independently selected from the Group One Substituents;
   (e) a 6- to 10-membered aromatic, optionally substituted with 1, 2, 3, or 4 substituents independently selected from the group consisting of:
      (1) phenyl;
      (2) halide;
      (3) cyano;
      (4) C1-C6 linear or branched alkyl, optionally substituted with
         (i) 1, 2, 3, 4, 5, 6, 7, 8, or 9 fluorine atoms; and/or
         (ii) 1, 2, or 3 substituents independently selected from the Group Two Substituents, and
      (5) C1-C6 linear or branched heteroalkyl containing 1, 2, or 3 atoms independently selected from O, N, and S and optionally substituted with
         (i) 1, 2, 3, 4, 5, 6, 7, 8, or 9 fluorine atoms; and/or
         (ii) 1, 2, or 3 substituents independently selected from the Group Two Substituents;
   (f) 5- to 10-membered heteroaromatic comprising 1, 2, 3, 4, 5, or 6 heteroatoms independently selected from O, N, and S and optionally substituted with 1, 2, 3, or 4 substituents independently selected from
      (1) phenyl;
      (2) halide;
      (3) cyano;
      (4) trifluoromethyl;
      (5) C1-C6 linear or branched alkyl optionally substituted with
         (i) 1, 2, 3, 4, 5, 6, 7, 8, or 9 fluorine atoms; and/or
         (ii) 1, 2, or 3 substituents independently selected from the Group Two Substituents; and
      (6) C1-C6 linear or branched heteroalkyl containing 1, 2, or 3 atoms independently selected from O, N, and S and optionally substituted with
         (i) 1, 2, 3, 4, 5, 6, 7, 8, or 9 fluorine atoms; and/or
         (ii) 1, 2, or 3 substituents independently selected from the Group Two Substituents;
   (g)

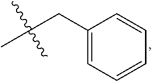 [SMILES: *Cc1ccccc1]

optionally substituted with 1, 2, or 3 substituents independently selected from the group consisting of:
   (1) C1-C6 linear or branched alkyl, optionally substituted with
      (i) 1, 2, 3, 4, 5, or 6 fluorine atoms; and/or
      (ii) 1, 2, or 3 substituents independently selected from the Group Two Substituents;
(h) 3- to 9-membered cycloheteroalkyl having 1, 2, or 3 heteroatoms independently selected from O, N, and S and optionally substituted with 1, 2, or 3 substituents independently selected from the group consisting of:
   (1) C1-C6 linear or branched alkyl, optionally substituted with
      (i) 1, 2, 3, 4, 5, 6, 7, 8, or 9 fluorine atoms; and/or
      (ii) 1, 2, or 3 substituents independently selected from the Group Two Substituents,
   (2) C1-C6 linear or branched heteroalkyl, optionally substituted with
      (i) 1, 2, 3, 4, 5, 6, 7, 8, or 9 fluorine atoms and/or
      (ii) 1, 2, or 3 substituents independently selected from the Group Two Substituents,
   (3) phenyl, optionally substituted with 1, 2, or 3 substituents independently selected from the Group Two Substituents, and
   (4) 5- to 10-membered heteroaromatic, optionally substituted with 1, 2, or 3 substituents independently selected from the Group Two Substituents; and
(i) C3-C6 cycloalkyl, optionally substituted with 1, 2, or 3 substituents independently selected from:
   (1) C1-C6 linear or branched alkyl, optionally substituted with
      (i) 1, 2, 3, 4, 5, 6, 7, 8, or 9 fluorine atoms; and/or
      (ii) 1, 2, or 3 substituents independently selected from the Group Two Substituents,
   (2) C1-C6 linear or branched heteroalkyl, optionally substituted with
      (i) 1, 2, 3, 4, 5, 6, 7, 8, or 9 fluorine atoms; and/or
      (ii) 1, 2, or 3 substituents independently selected from the Group Two Substituents,
   (3) phenyl, optionally substituted with 1, 2, or 3 substituents independently selected from Group Two Substituents; and
   (4) 5- to 10-membered heteroaromatic, optionally substituted with 1, 2, or 3 substituents independently selected from the Group Two Substituents.

### Cannabinoid Component

A “cannabinoid component” as used in this disclosure is that portion of a cannabinoid that is present in the conjugate molecule and covalently attached to a linker, as shown in the examples below.

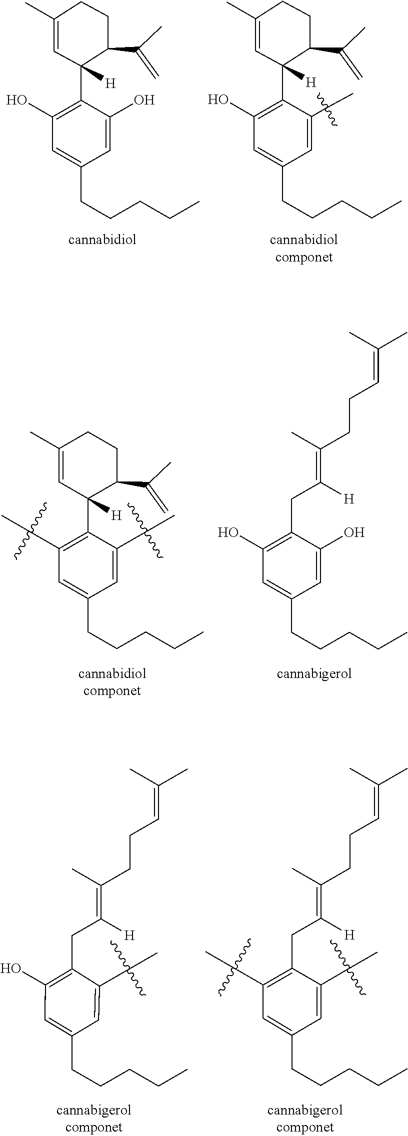 [SMILES: C=C(C)[C@@H]1CCC(C)=C[C@H]1c1c(O)cc(CCCCC)cc1O ; *c1cc(CCCCC)cc(O)c1[C@@H]1C=C(C)CC[C@H]1C(=C)C ; *c1cc(CCCCC)cc(*)c1[C@@H]1C=C(C)CC[C@H]1C(=C)C ; CCCCCc1cc(O)c(C/C=C(\C)CCC=C(C)C)c(O)c1 ; *c1cc(CCCCC)cc(O)c1C/C=C(\C)CCC=C(C)C ; *c1cc(CCCCC)cc(*)c1C/C=C(\C)CCC=C(C)C]

The cannabinoid component can be provided by any cannabinoid that contains a hydroxy group to which the linker can be attached or to which an avermectin component can be covalently attached or a carboxylic acid to which a linker can be connected by way of an ester, amide, or thioester bond. The cannabinoid can be a naturally occurring molecule, either isolated or synthesized, or a modified version of a naturally occurring molecule. See, for example, Morales et al., Frontiers in Pharmacology June 2017 review, 1-18.

Examples of cannabinoids include, but are not limited to, cannabigerols, cannabichromenes, cannabidiols, tetrahydrocannabinols, cannabicyclols, cannabielsoins, cannabinols, cannabinodiols, cannabitriols, dehydrocannabifurans, cannabifurans, cannabichromanons, and cannabiripsols.

Examples of cannabigerols include cannabigerolic acid (CBGA), cannabigerolic acid monomethylether (CBGAM), cannabigerol (CBG), cannabigerol monomethyleither (CBGM), cannabigerovarinic acid (CBGVA), and cannabigerovarin (CBGV).

Examples of cannabichromenes include cannabichromenic acid (CBC), cannabichromene (CBC), cannabichromevarinic acid (CBCVA), and cannabichromevarin (CBCV).

Examples of cannabidiols include cannabidiolic acid (CBDA), cannabidiol (CBD), cannabidiol monomethylether (CBDM), cannabidiol-C₄ (CBD-C₄), cannabidivarinic acid (CBDVA), cannabidivarin (CBDV), and cannabidiorcol (CBD-C1).

Examples of tetrahydrocannabinols include Δ-9-tetrahydrocannabinolic acid A (THCA-A), Δ-9-tetrahydrocannabinolic acid B (THCA-B), Δ-9-tetrahydrocannabinol (THC), Δ-9-tetrahydrocannabinolic acid-C₄ (THCA-C₄), Δ-9-tetrahydrocannabinol-C₄ (THC-C₄), Δ-9-tetrahydrocannabivarinic acid (THCVA), Δ-9-tetrahydrocannabivarin (THCV), Δ-9-tetrahydrocannabiorcolic acid (THCA-C₁), Δ-9-tetrahydrocannabiorcol (THC-C₁), Δ-7-cis-tetrahydrocannabivarin, Δ-8-tetrahydrocannabinolic acid (Δ⁸-THCA), and Δ-8-tetrahydrocannabinol (Δ⁸-THC).

Examples of cannabicyclols include cannabicyclolic acid (CBLA), cannabicyclol (CBL), and cannabicyclovarin (CBLV).

Examples of cannabielsoins include cannabielsoic acid A (CBEA-A), cannabielsoic acid B (CBEA-B), and cannabielsoin (CBE).

Examples of cannabinols and cannabinodiols include cannabinolic acid (CBNA), cannabinol (CBN), cannabinol-C₄ (CBN-C₄), cannabivarin (CBV), cannabinol-C₂ (CBN-C₂), cannabiorcol (CBN-C₁), cannabinodiol (CBND), and cannabinodivarin (CBVD).

Examples of cannabitriols include cannabitriol (CBT), 10-ethoxy-9-hydroxy-Δ-6a-tetrahydrocannabinol, cannabitriolvarin (CBTV), and ethoxy-cannabitriolvarin (CBTVE).

Cannabifurans include dehydrocannabifuran (DCBF) and cannabifuran (CBF).

Examples of other cannabinoids include cannabichromanon (CBCN), 10-oxo-Δ-6a-tetrahydrocannabinol (OTHC), cannabiripsol (CBR), and trihydroxy-Δ-9-tetrahydrocannabinol (triOH-THC).

In some embodiments, the cannabinoid component is provided by cannabidiol.

### Conjugate Molecules Comprising Two Avermectin Components

In some embodiments, in which the cannabinoid component has two hydroxyl groups, a second avermectin component can be covalently attached to the second hydroxyl group by means of a second linker such that the conjugate molecule contains a first avermectin component and a second avermectin component covalently attached to the cannabinoid component by means of a first linker and a second linker, respectively.

Conjugate molecules in which at least one of the linkers is

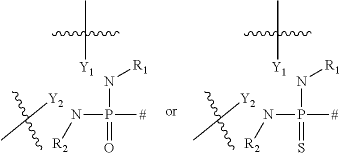

can comprise a second avermectin component covalently attached to the linker rather than to the cannabinoid component. In some embodiments, first avermectin component is covalently attached at Y₂. In some embodiments, the first avermectin component is covalently attached at Y₁.

In conjugate molecules comprising two avermectin components, the avermectin components can be the same or different.

### Conjugate Molecules Comprising Two or Three Cannabinoid Components

Conjugate molecules comprising a first avermectin component can comprise one, two, or three cannabinoid components.

In some embodiments, a conjugate molecule comprises a first avermectin component, a first cannabinoid component, and a second cannabinoid component. The first and second cannabinoid components can be the same or different,

In some embodiments, a conjugate molecule comprises a first cannabinoid component, a second cannabinoid component, and a third cannabinoid component. In some embodiments, the first and second cannabinoid components are the same and the third cannabinoid component is different. In some embodiments, the second and third cannabinoid components are the same and the first cannabinoid component is different. In some embodiments, the first and third cannabinoid components are the same and the second cannabinoid component is different. In some embodiments, the first, second, and third cannabinoid components are the same. In some embodiments, the first, second, and third cannabinoid components are different.

In any embodiments comprising two or three cannabinoid components, each cannabinoid component can be the same or different, and, when linkers are used, each linker can be the same or different.

### Examples of Conjugate Molecules

The following Examples show a conjugate with either one or two linked cannabinoids. Any of the three OH groups may be linked in the general fashion shown in these Examples using linkers as defined.

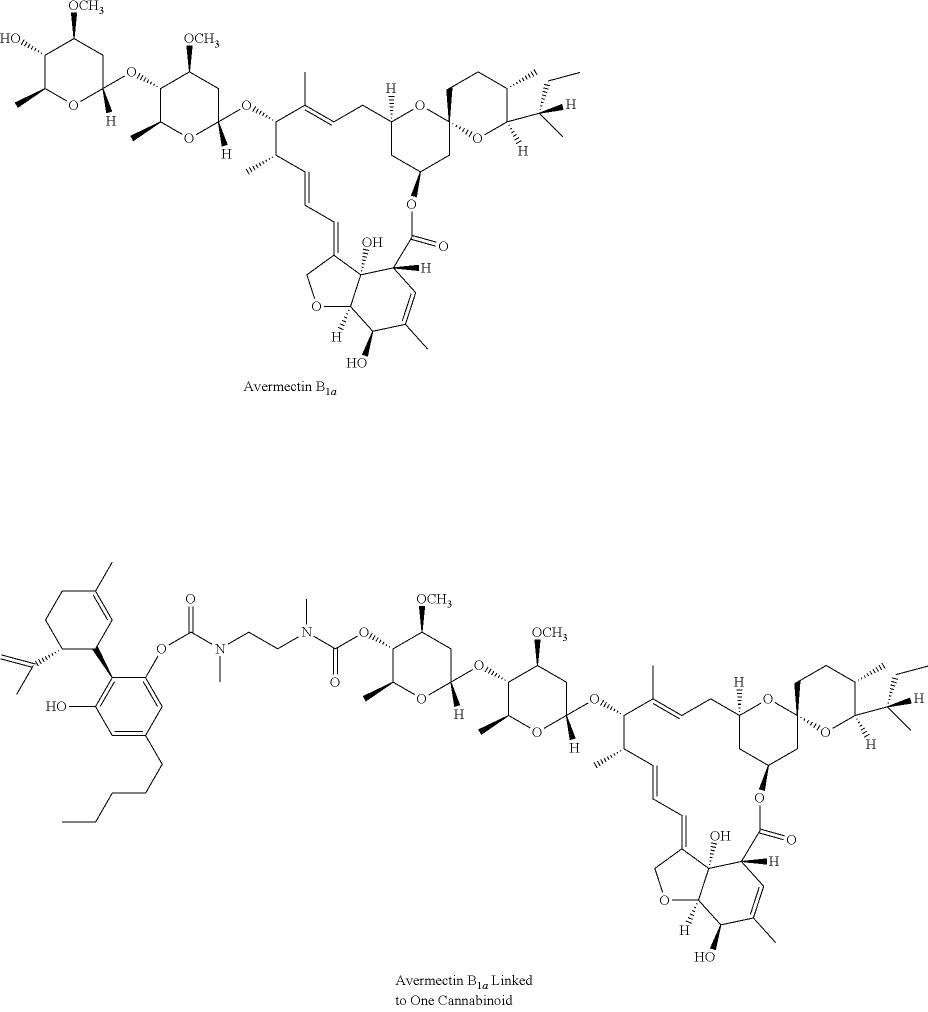

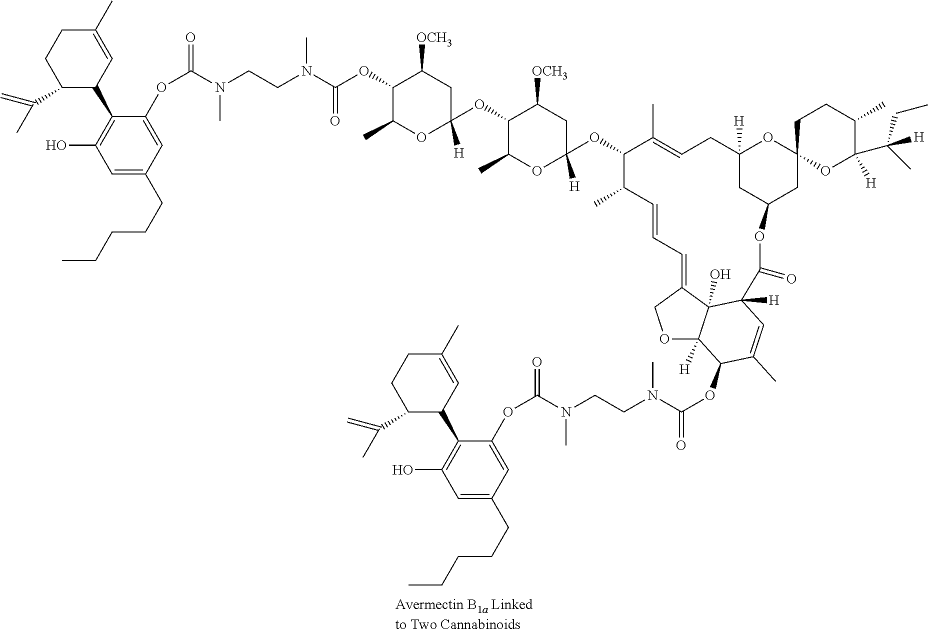

The following Examples show a conjugate with either one or two linked cannabinoids. Any of the three OH groups may be linked in the general fashion shown in these Examples using linkers as defined.

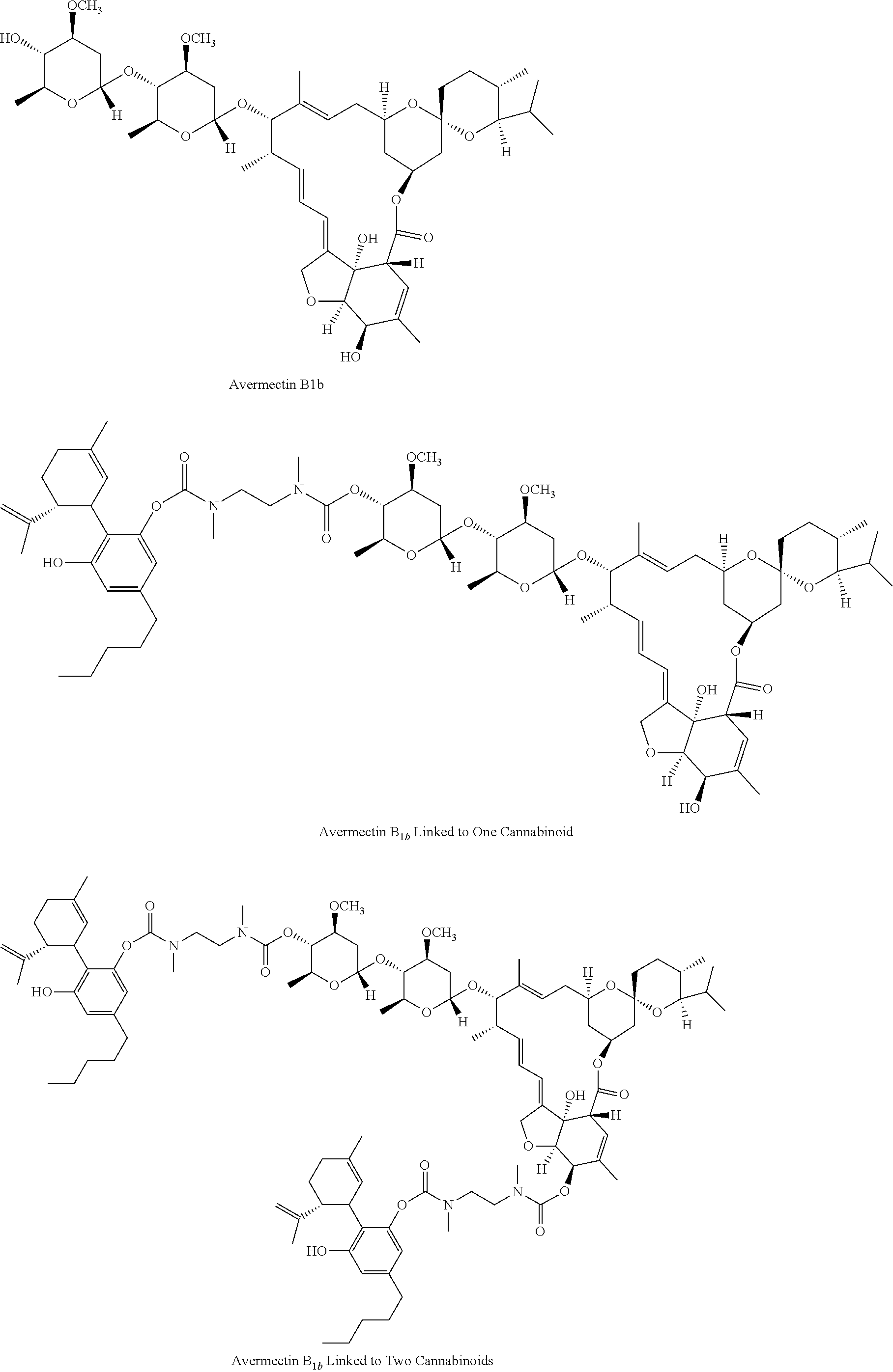

In the example below, two molecules of avermectin B₁ₐ are conjugated to one cannabinoid molecule.

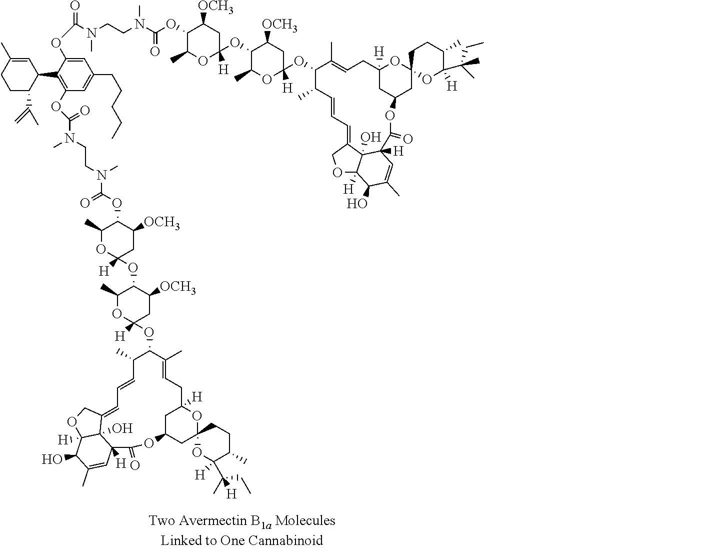

In the example below, two molecules of avermectin B_{1b} are conjugated to one cannabinoid molecule.

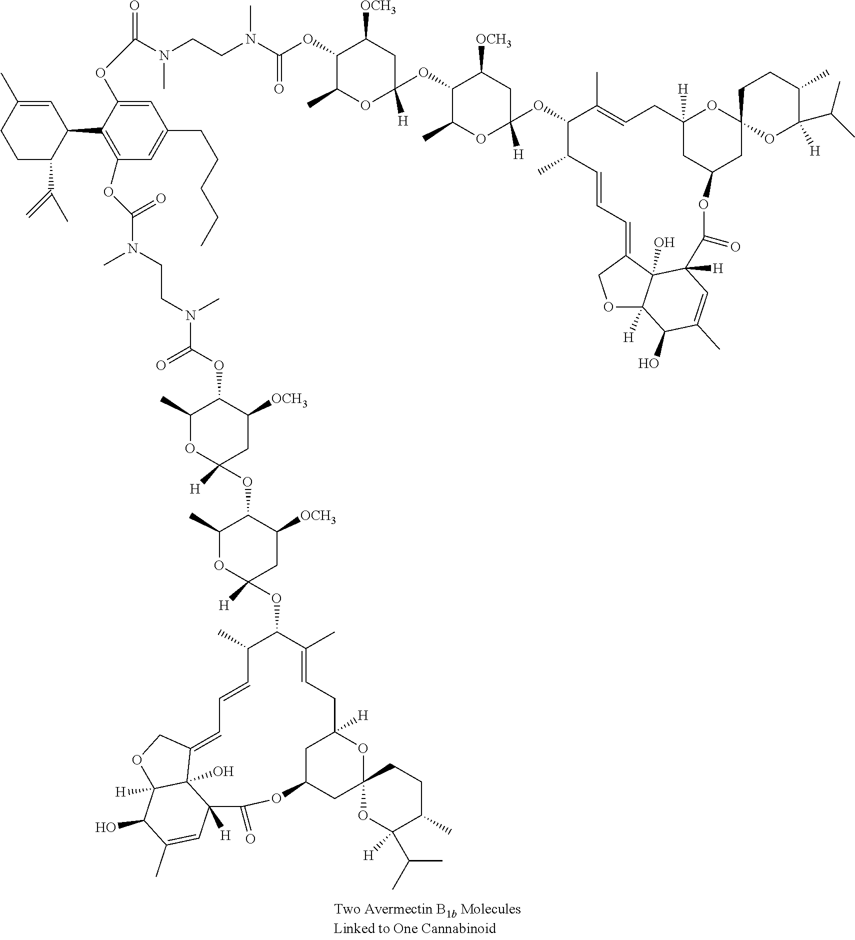 [SMILES: C=C(C)[C@@H]1CCC(C)=C[C@H]1c1c(OC(=O)N(C)CCN(C)C(=O)O[C@H]2[C@H](C)O[C@@H](O[C@H]3[C@H](C)O[C@@H](O[C@@H]4/C(C)=C/C[C@@H]5C[C@@H](C[C@]6(CC[C@H](C)[C@@H](C(C)C)O6)O5)OC(=O)[C@@H]5C=C(C)[C@@H](O)[C@H]6OC/C(=C\C=C\[C@@H]4C)[C@]65O)C[C@@H]3OC)C[C@@H]2OC)cc(CCCCC)cc1OC(=O)N(C)CCN(C)C(=O)O[C@H]1[C@H](C)O[C@@H](O[C@H]2[C@H](C)O[C@@H](O[C@@H]3/C(C)=C/C[C@@H]4C[C@@H](C[C@]5(CC[C@H](C)[C@@H](C(C)C)O5)O4)OC(=O)[C@@H]4C=C(C)[C@@H](O)[C@H]5OC/C(=C\C=C\[C@@H]3C)[C@]54O)C[C@@H]2OC)C[C@@H]1OC]

In the example below, one cannabinoid is linked to one molecule of avermectin B₁ₐ and one molecule of avermectin B_{1b}.

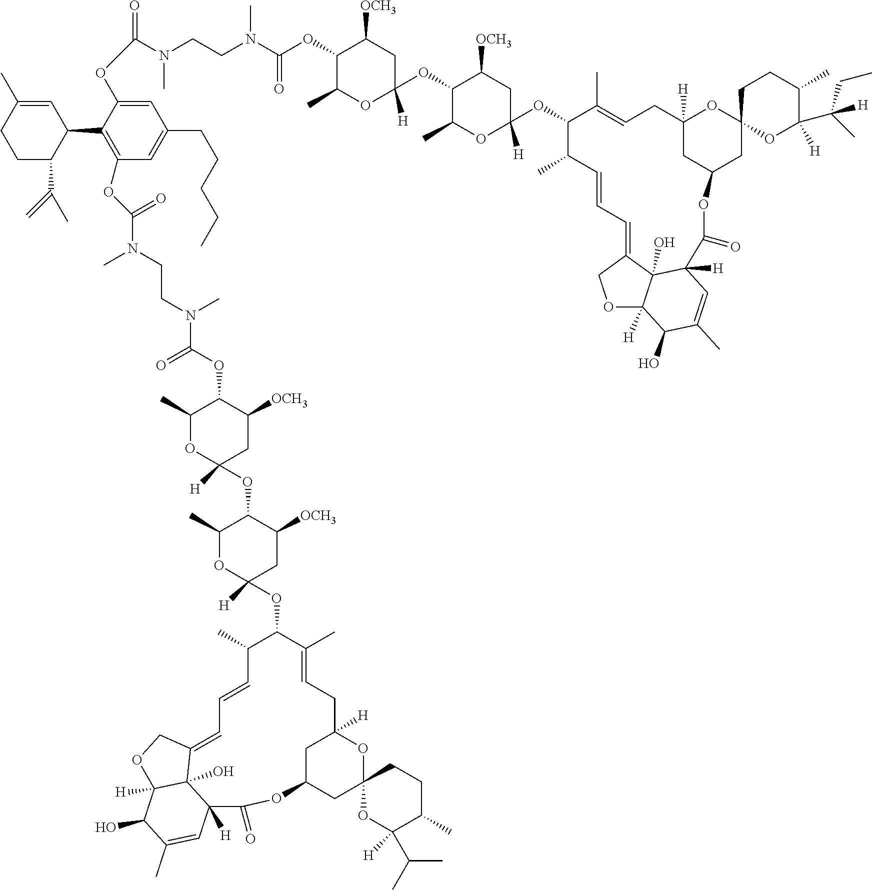 [SMILES: C=C(C)[C@@H]1CCC(C)=C[C@H]1c1c(OC(=O)N(C)CCN(C)C(=O)O[C@H]2[C@H](C)O[C@@H](O[C@H]3[C@H](C)O[C@@H](O[C@@H]4/C(C)=C/C[C@@H]5C[C@@H](C[C@]6(CC[C@H](C)[C@@H]([C@@H](C)CC)O6)O5)OC(=O)[C@@H]5C=C(C)[C@@H](O)[C@H]6OC/C(=C\C=C\[C@@H]4C)[C@]65O)C[C@@H]3OC)C[C@@H]2OC)cc(CCCCC)cc1OC(=O)N(C)CCN(C)C(=O)O[C@H]1[C@H](C)O[C@@H](O[C@H]2[C@H](C)O[C@@H](O[C@@H]3/C(C)=C/C[C@@H]4C[C@@H](C[C@]5(CC[C@H](C)[C@@H](C(C)C)O5)O4)OC(=O)[C@@H]4C=C(C)[C@@H](O)[C@H]5OC/C(=C\C=C\[C@@H]3C)[C@]54O)C[C@@H]2OC)C[C@@H]1OC]

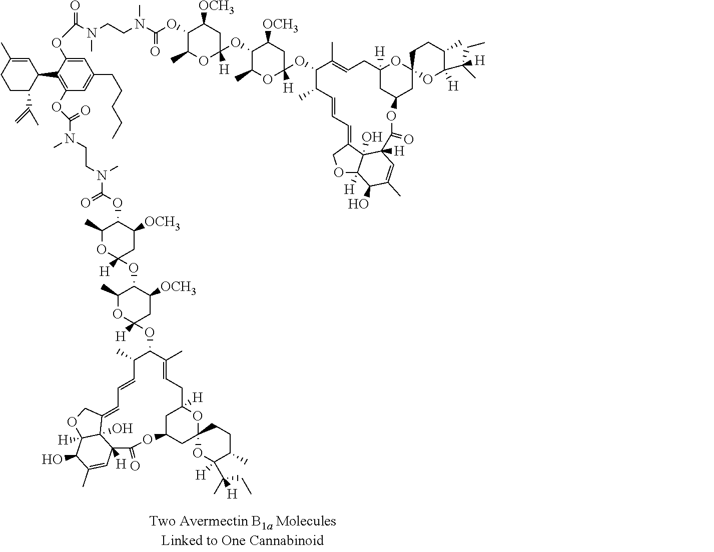 [SMILES: C=C(C)[C@@H]1CCC(C)=C[C@H]1c1c(OC(=O)N(C)CCN(C)C(=O)O[C@H]2[C@H](C)O[C@@H](O[C@H]3[C@H](C)O[C@@H](O[C@@H]4/C(C)=C/C[C@@H]5C[C@@H](C[C@]6(CC[C@H](C)[C@@H]([C@@H](C)CC)O6)O5)OC(=O)[C@@H]5C=C(C)[C@@H](O)[C@H]6OC/C(=C\C=C\[C@@H]4C)[C@]65O)C[C@@H]3OC)C[C@@H]2OC)cc(CCCCC)cc1OC(=O)N(C)CCN(C)C(=O)O[C@H]1[C@H](C)O[C@@H](O[C@H]2[C@H](C)O[C@@H](O[C@@H]3/C(C)=C/C[C@@H]4C[C@@H](C[C@]5(CC[C@H](C)[C@@H]([C@@H](C)CC)O5)O4)OC(=O)[C@@H]4C=C(C)[C@@H](O)[C@H]5OC/C(=C\C=C\[C@@H]3C)[C@]54O)C[C@@H]2OC)C[C@@H]1OC]

### Compositions, Routes of Administration, and Dosages

One or more conjugate molecules, which can be the same or different, can be provided in a composition, e.g., a pharmaceutical composition, together with a pharmaceutically acceptable vehicle. The “pharmaceutically acceptable vehicle” can comprise one or more substances which do not affect the biological activities of the conjugate molecules and, when administered to a patient, do not cause an adverse reaction. Excipients, such as calcium carbonate, calcium phosphate, various sugars and types of starch, cellulose derivatives, and gelatin can be included. Pharmaceutically acceptable vehicles for liquid compositions include, but are not limited to, water, saline, polyalkylene glycols (e.g., polyethylene glycol), vegetable oils, and hydrogenated naphthalenes. Controlled release, for example, can be achieved using biocompatible, biodegradable polymers of lactide or copolymers of lactide/glycolide or polyoxyethylene/polyoxypropylene.

Methods of preparing pharmaceutical compositions are well known. Pharmaceutical compositions can be prepared as solids, semi-solids, or liquid forms, such as tablets, capsules, powders, granules, ointments, solutions, suspensions, emulsions, suppositories, injections, inhalants, gels, microspheres, aerosols, and mists. Liquid pharmaceutical compositions can be lyophilized. Lyophilized compositions can be provided in a kit with a suitable liquid, typically water for injection (WFI) for use in reconstituting the composition.

Typical administration routes include, but are not limited to, oral, topical, transdermal, inhalation, parenteral, sublingual, buccal, rectal, vaginal, and intranasal.

The dose of a pharmaceutical composition can be based on the doses typically used for the particular avermectin agent(s) which provide the avermectin component(s) of a conjugate molecule. These doses are well known in the art.

In a composition containing more than one type of conjugate molecule disclosed herein, the portion of any type of conjugate molecule may be from 1 to 99 percent. For example, in a composition comprising a mixture of conjugates avermectin B₁ₐ and B_{1b}, the ratio of the B₁ₐ and B_{1b} conjugates can be approximately 75:25 to 95:5.

### Therapeutic Methods

The disclosed conjugate molecules have a variety of therapeutic uses depending on which avermectin component(s) are included in a conjugate molecule. “Treat” as used in this disclosure means reducing or inhibiting the progression of one or more symptoms of the disorder or disease for which the conjugate molecule is administered, such as inflammation or pain.

Avermectins have anthelmintic and insecticidal properties and can be used to treat pests and parasitic worms (e.g., trematodes, cestodes, and nematodes). Avermectins also have been reported to have anti-cancer, anti-diabetic, anti-fungal, and anti-inflammatory properties and are used to treat several metabolic disorders. See Batiha and see Liu et al., Drug Design, and Development and Therapy 14, 285-296, 2020. Anti-viral activity has been reported, for example, for ivermectin; see Martin et al., Trends in Parasitology 37(1), 48-64, 2021 (summarizing experiments demonstrating activity in vitro against the RNA viruses dengue virus, West Nile virus, and Venezuelan equine encephalitis virus; activity in vitro and in vivo against the DNA virus pseudorabies virus; and, at high concentrations, activity in vitro against SARS-COV-2-infected Vero/hSLAM cells). Anti-tumor activity has also been reported, for example, for example, for ivermectin; see Liu, 2020 (summarizing experiments demonstrating activity in vitro against multiple cancers including breast, colon, gastric, glioblastoma, leukemia, melanoma, and ovarian). An advantage of conjugate molecules is that a cannabinoid can be delivered directly to the site of action of the avermectin agent, where the released cannabinoid can provide further therapeutic benefits. The therapeutic benefits and potential benefits of cannabinoids are well known. For example, see Dzierzanowski, Cancers 11, 129-41, 2019 (oncology and palliative care); Urits et al., Pain Ther. 8, 41-51, 2019 (pain); Hillen et al., Ther. Adv. Drug Safety 10, 1-23 2019 (neuropsychiatric symptoms in dementia). Accordingly, the disclosed conjugate molecules may be useful for treating infections caused by viruses, parasitic worms, and fungi.

### EXAMPLES

The following procedures for synthesizing various types and classes of compounds are general representative procedures for building in the primary functionality of the compounds. The reagent system, reaction conditions, and protecting group strategy may vary for any specific analog. Specific building blocks vary in accordance with the specific desired product. The Example below shows cannabidiol (CBD) as a representative cannabinoid, although other cannabinoids containing hydroxyl groups may be substituted to generate alternative analogs.

### Example 1

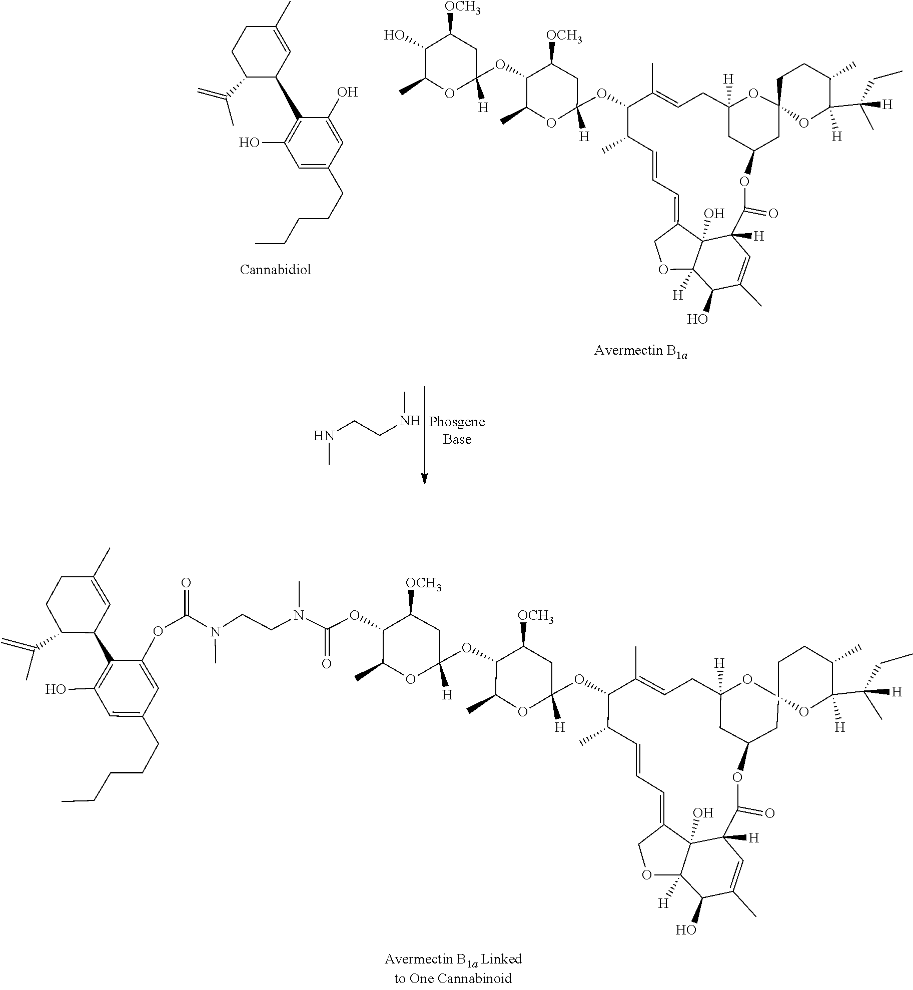

Example 1 may be synthesized by reacting cannabidiol and avermectin B₁ₐ with dimethylethylamine and phosgene as shown in the Scheme. Protecting groups known in the field of synthetic organic chemistry may be employed as needed on any of the building blocks to facilitate the synthesis. Phosgene is shown as an example of a carbamate forming reagent, but other methods in the field of organic chemistry for synthesizing carbamates may be substituted for the phosgene reagent system shown above. Although the Scheme shows a one step process, the actual synthesis may employ multiple steps due to the use of protecting groups, as well as a stepwise formation of the two carbamate functions whereby the carbamate is first formed between the diamino building block and the avermectin B₁ₐ to generate a synthetic intermediate which is subsequently reacted with cannabidiol (or a protected derivative). The order of carbamate formation may also be reversed, with the cannabidiol moiety being first reacted with the diamino building block to form a synthetic intermediate which is subsequently reacted with avermectin B₁ₐ. Synthetic intermediates and the final product may be purified using standard purification methods in the field of synthetic organic chemistry.

Other Examples shown in this Application or envisioned within the scope of the definitions are synthesized via related strategies. The synthetic routes may include protecting groups as needed to facilitate the preparation or purification of synthetic intermediates, and reagent systems known in the field of organic chemistry are employed for creating the linking functional groups described in the definitions herein.

1. A conjugate molecule, comprising a first avermectin component and a first cannabinoid component, wherein a first avermectin attachment point is covalently attached via a first linker to a first cannabinoid attachment point of the first cannabinoid component.

2. The conjugate molecule of claim 1, further comprising a second cannabinoid component covalently attached via a second linker to a second avermectin attachment point of the first avermectin component.

3. The conjugate molecule of claim 2, further comprising a third cannabinoid component covalently attached via a third linker to a third avermectin attachment point of the first avermectin component.

4. The conjugate molecule of claim 1, further comprising a second avermectin component covalently attached via a second linker to a second cannabinoid attachment point of the first cannabinoid component.

5. The conjugate molecule of claim 1, wherein the first avermectin component is provided by avermectin, ivermectin, selamectin, doramectin, eprinomectin, emamectin, or moxidectin or an active metabolite thereof.

6. The conjugate molecule of claim 4, wherein the first and second avermectin components independently are provided by avermectin, ivermectin, selamectin, doramectin, eprinomectin, emamectin, or moxidectin or active metabolites thereof.

7. The conjugate molecule of claim 1, wherein the first cannabinoid component is provided by a cannabinoid selected from the group consisting of a cannabigerol, a cannabichromene, a cannabidiol, a tetrahydrocannabinol, a cannabicyclol, a cannabielsoin, a cannabinol, a cannabinodiol, a cannabitriol, a dehydrocannabifuran, a cannabifuran, a cannabichromanon, and a cannabiripsol, or an active metabolite thereof.

8. The conjugate molecule of claim 2, wherein the first and second cannabinoid components independently are provided by a cannabinoid selected from the group consisting of a cannabigerol, a cannabichromene, a cannabidiol, a tetrahydrocannabinol, a cannabicyclol, a cannabielsoin, a cannabinol, a cannabinodiol, a cannabitriol, a dehydrocannabifuran, a cannabifuran, a cannabichromanon, and a cannabiripsol, or an active metabolite thereof.

9. The conjugate molecule of claim 3, wherein the first, second, and third cannabinoid components independently are provided by a cannabinoid selected from the group consisting of a cannabigerol, a cannabichromene, a cannabidiol, a tetrahydrocannabinol, a cannabicyclol, a cannabielsoin, a cannabinol, a cannabinodiol, a cannabitriol, a dehydrocannabifuran, a cannabifuran, a cannabichromanon, and a cannabiripsol, or an active metabolite thereof.

10. The conjugate molecule of claim 1, wherein the first linker is selected from the group consisting of: 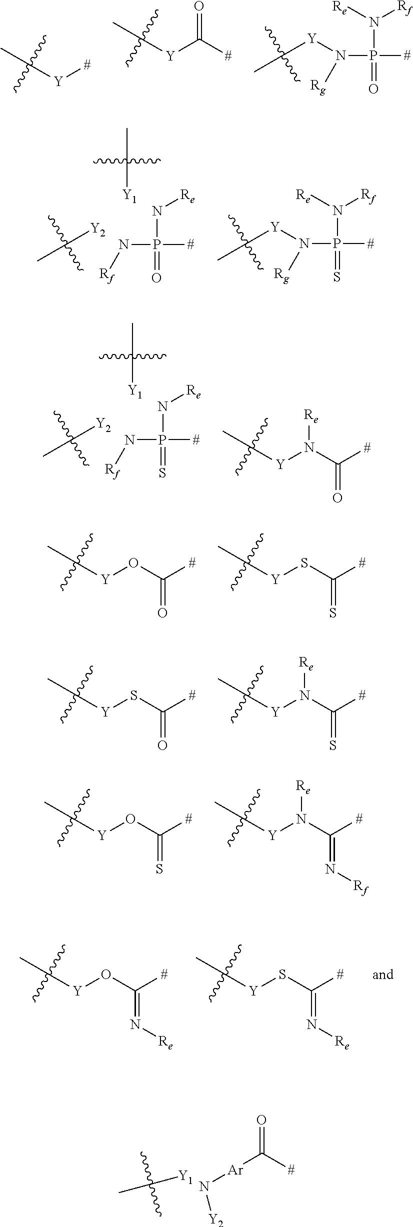
in which [symbol] marks a bond attaching the first linker to the first avermectin component, # indicates a site of covalent attachment to the first cannabinoid component, and in which:
Y, Y₁, and Y₂ independently are absent or Y, Y₁, and Y₂ independently are selected from the group consisting of:
(a) C1-C12 linear or branched alkyl, optionally substituted with
(1) 1, 2, 3, 4, 5, 6, 7, 8, or 9 fluorine atoms; and/or
(2) 1, 2, or 3 substituents selected from the Group One Substituents;
(b) C2-C12 linear or branched alkenyl, optionally substituted with
(1) 1, 2, 3, 4, 5, 6, 7, 8, or 9 fluorine atoms; and/or
(2) 1, 2, or 3 substituents selected from the Group One Substituents;
(c) C1-C12 linear or branched heteroalkyl containing 1, 2, 3, or 4 heteroatoms independently selected from O, N, and S, optionally substituted with
(1) 1, 2, 3, 4, 5, 6, 7, 8, or 9 fluorine atoms; and/or
(2) 1, 2, or 3 substituents selected from the Group One Substituents;
(d) a 6- to 10-membered aromatic, optionally substituted with 1, 2, 3, or 4 substituents independently selected from the group consisting of:
(1) phenyl,
(2) halide,
(3) C1-C6 linear or branched alkyl, optionally substituted with
(i) 1, 2, 3, 4, 5, 6, 7, 8, or 9 fluorine atoms; and/or
(ii) 1, 2, or 3 substituents independently selected from the Group Two Substituents, and
(4) C1-C6 linear or branched heteroalkyl containing 1, 2, or 3 atoms independently selected from O, N, and S and optionally substituted with
(i) 1, 2, 3, 4, 5, 6, 7, 8, or 9 fluorine atoms; and/or
(ii) 1, 2, or 3 substituents independently selected from the Group Two Substituents;
(e) a 6- to 10-membered heteroaromatic comprising 1, 2, 3, 4, 5, or 6 heteroatoms independently selected from O, N, and S and optionally substituted with 1, 2, 3, or 4 substituents independently selected from
(1) phenyl,
(2) halide,
(3) trifluoromethyl,
(4) C1-C6 linear or branched alkyl, optionally substituted with
(i) 1, 2, 3, 4, 5, 6, 7, 8, or 9 fluorine atoms; and/or
(ii) 1, 2, or 3 substituents independently selected from the Group Two Substituents, and
(5) C1-C6 linear or branched heteroalkyl containing 1, 2, or 3 atoms independently selected from O, N, and S and optionally substituted with
(i) 1, 2, 3, 4, 5, 6, 7, 8, or 9 fluorine atoms; and/or
(ii) 1, 2, or 3 substituents independently selected from the Group Two Substituents; and
(f) a C1-C24 linear or branched heteroalkyl containing 1, 2, 3, 4, 5, 6, 7, or 8 heteroatoms independently selected from O, N, and S, optionally substituted with
(i) 1, 2, 3, 4, 5, 6, 7, 8, or 9 fluorine atoms; and/or
(ii) 1, 2, 3, 4, 5, or 6 substituents selected from the Group One Substituents; and
wherein Group One Substituents are is a group of substituents consisting of:
(a) —OH;
(b) —NH₂;
(c) ═O;
(d) ═S;
(e) ═NR7, where R7 is H or is C1-C3 linear or branched alkyl or C1-C3 linear or branched heteroalkyl comprising an O, N, or S atom;
(f) —C(O)OR4, wherein R4 is H or C1-C3 linear or branched alkyl;
(g) —C(O)NR5R6, wherein R5 and R6 independently are H or C1-C6 linear or branched alkyl;
(h) halide;
(i) C1-C6 linear or branched alkoxyl;
(j) C1-C6 linear or branched alkylamino;
(k) C1-C6 linear or branched dialkylamino;
(l) 6- to 10-membered aromatic, optionally substituted with 1, 2, 3, or 4 substituents independently selected from
(i) phenyl;
(ii) halide;
(iii) cyano;
(iv) C1-C6 linear or branched alkyl, optionally substituted with
(1) 1, 2, 3, 4, 5, 6, 7, 8, or 9 fluorine atoms; and/or
(2) 1, 2, or 3 substituents independently selected from the Group Two Substituents; and
(v) C1-C6 linear or branched heteroalkyl containing 1, 2, or 3 atoms independently selected from O, N, and S and optionally substituted with
(1) 1, 2, 3, 4, 5, 6, 7, 8, or 9 fluorine atoms; and/or
(2) 1, 2, or 3 substituents independently selected from the Group Two Substituents;
(m) 5- to 10-membered heteroaromatic, optionally substituted with 1, 2, 3, or 4 substituents independently selected from
(i) phenyl;
(ii) halide;
(iii) cyano;
(iv) C1-C6 linear or branched alkyl, optionally substituted with
(1) 1, 2, 3, 4, 5, 6, 7, 8, or 9 fluorine atoms; and/or
(2) 1, 2, or 3 substituents independently selected from the Group Two Substituents; and
(v) C1-C6 linear or branched heteroalkyl containing 1, 2, or 3 atoms independently selected from O, N, and S and optionally substituted with
(1) 1, 2, 3, 4, 5, 6, 7, 8, or 9 fluorine atoms; and/or
(2) 1, 2, or 3 substituents independently selected from the Group Two Substituents;
(n) 3- to 9-membered cycloheteroalkyl having 1, 2, or 3 heteroatoms independently selected from O, N, and S, optionally substituted with 1, 2, 3, or 4 substituents independently selected from
(i) phenyl;
(ii) halide;
(iii) cyano;
(iv) C1-C6 linear or branched alkyl, optionally substituted with
(1) 1, 2, 3, 4, 5, 6, 7, 8, or 9 fluorine atoms; and/or
(2) 1, 2, or 3 substituents independently selected from the Group Two Substituents; and
(v) C1-C6 linear or branched heteroalkyl containing 1, 2, or 3 atoms independently selected from O, N, and S and optionally substituted with
(1) 1, 2, 3, 4, 5, 6, 7, 8, or 9 fluorine atoms; and/or
(2) 1, 2, or 3 substituents independently selected from the Group Two Substituents; and
(o) C3-C6 cycloalkyl, optionally substituted with 1, 2, 3, or 4 substituents independently selected from
(i) phenyl;
(ii) halide;
(iii) cyano;
(iv) C1-C6 linear or branched alkyl, optionally substituted with
(1) 1, 2, 3, 4, 5, 6, 7, 8, or 9 fluorine atoms; and/or
(2) 1, 2, or 3 substituents independently selected from the Group Two Substituents; and
(v) C1-C6 linear or branched heteroalkyl containing 1, 2, or 3 atoms independently selected from O, N, and S and optionally substituted with
(1) 1, 2, 3, 4, 5, 6, 7, 8, or 9 fluorine atoms; and/or
(2) 1, 2, or 3 substituents independently selected from the Group Two Substituents; and
wherein Group Two Substituents are is a group of substituents consisting of:
(a) —OH;
(b) —NH₂;
(c) ═O;
(d) ═S;
(e) ═NR7, where R7 is H or is C1-C3 linear or branched alkyl or C1-C3 linear or branched heteroalkyl comprising an O, N, or S atom;
(f) —C(O)OR4, wherein R4 is H or C1-C3 linear or branched alkyl;
(g) —C(O)NR5R6, wherein R5 and R6 independently are H or C1-C6 linear or branched alkyl;
(h) halide;
(i) cyano;
(j) trifluoromethyl;
(k) C1-C6 linear or branched alkoxyl;
(l) C1-C6 linear or branched alkylamino;
(m) C1-C6 linear or branched dialkylamino;
(n) 6- to 10-membered aromatic; and
(o) 5- to 10-membered heteroaromatic comprising 1, 2, 3, 4, 5, or 6 heteroatoms independently selected from O, N, and S; and
Rₑ, R_{f}, and R_{g} independently are selected from the group consisting of:
(a) H;
(b) C1-C8 linear or branched alkyl, optionally substituted with
(1) 1, 2, 3, 4, 5, 6, 7, 8, or 9 fluorine atoms; and/or
(2) 1, 2, or 3 substituents independently selected from the Group One Substituents;
(c) C1-C8 linear or branched heteroalkyl containing 1, 2, or 3 heteroatoms independently selected from O, N, and S and optionally substituted with
(1) 1, 2, 3, 4, 5, 6, 7, 8, or 9 fluorine atoms; and/or
(2) 1, 2, or 3 substituents independently selected from the Group One Substituents;
(d) phenyl, optionally substituted with 1, 2, or 3 substituents independently selected from the group consisting of:
(1) C1-C6 linear or branched alkyl, optionally substituted with
(i) 1, 2, 3, 4, 5, or 6 fluorine atoms; and/or
(ii) 1 or 2 substituents independently selected from the Group Two Substituents; and
(2) C1-C6 linear or branched heteroalkyl containing 1 or 2 heteroatoms independently selected from O, N, and S and optionally substituted with
(i) 1, 2, 3, 4, 5, or 6 fluorine atoms; and/or
(ii) 1 or 2 substituents independently selected from the Group One Substituents;
(e) a 6- to 10-membered aromatic, optionally substituted with 1, 2, 3, or 4 substituents independently selected from the group consisting of:
(1) phenyl;
(2) halide;
(3) cyano;
(4) C1-C6 linear or branched alkyl, optionally substituted with
(i) 1, 2, 3, 4, 5, 6, 7, 8, or 9 fluorine atoms; and/or
(ii) 1, 2, or 3 substituents independently selected from the Group Two Substituents, and
(5) C1-C6 linear or branched heteroalkyl containing 1, 2, or 3 atoms independently selected from O, N, and S and optionally substituted with
(i) 1, 2, 3, 4, 5, 6, 7, 8, or 9 fluorine atoms; and/or
(ii) 1, 2, or 3 substituents independently selected from the Group Two Substituents;
(f) 5- to 10-membered heteroaromatic comprising 1, 2, 3, 4, 5, or 6 heteroatoms independently selected from O, N, and S and optionally substituted with 1, 2, 3, or 4 substituents independently selected from
(1) phenyl;
(2) halide;
(3) cyano;
(4) trifluoromethyl;
(5) C1-C6 linear or branched alkyl optionally substituted with
(i) 1, 2, 3, 4, 5, 6, 7, 8, or 9 fluorine atoms; and/or
(ii) 1, 2, or 3 substituents independently selected from the Group Two Substituents; and
(6) C1-C6 linear or branched heteroalkyl containing 1, 2, or 3 atoms independently selected from O, N, and S and optionally substituted with
(i) 1, 2, 3, 4, 5, 6, 7, 8, or 9 fluorine atoms; and/or
(ii) 1, 2, or 3 substituents independently selected from the Group Two Substituents;
(g) 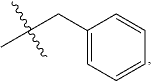 [SMILES: *Cc1ccccc1]
optionally substituted with 1, 2, or 3 substituents independently selected from the group consisting of:
(1) C1-C6 linear or branched alkyl, optionally substituted with
(i) 1, 2, 3, 4, 5, or 6 fluorine atoms; and/or
(ii) 1, 2, or 3 substituents independently selected from the Group Two Substituents;
(h) 3- to 9-membered cycloheteroalkyl having 1, 2, or 3 heteroatoms independently selected from O, N, and S and optionally substituted with 1, 2, or 3 substituents independently selected from the group consisting of:
(1) C1-C6 linear or branched alkyl, optionally substituted with
(i) 1, 2, 3, 4, 5, 6, 7, 8, or 9 fluorine atoms; and/or
(ii) 1, 2, or 3 substituents independently selected from the Group Two Substituents,
(2) C1-C6 linear or branched heteroalkyl, optionally substituted with
(i) 1, 2, 3, 4, 5, 6, 7, 8, or 9 fluorine atoms and/or
(ii) 1, 2, or 3 substituents independently selected from the Group Two Substituents,
(3) phenyl, optionally substituted with 1, 2, or 3 substituents independently selected from the Group Two Substituents, and
(4) 5- to 10-membered heteroaromatic, optionally substituted with 1, 2, or 3 substituents independently selected from the Group Two Substituents; and
(i) C3-C6 cycloalkyl, optionally substituted with 1, 2, or 3 substituents independently selected from:
(1) C1-C6 linear or branched alkyl, optionally substituted with
(i) 1, 2, 3, 4, 5, 6, 7, 8, or 9 fluorine atoms; and/or
(ii) 1, 2, or 3 substituents independently selected from the Group Two Substituents,
(2) C1-C6 linear or branched heteroalkyl, optionally substituted with
(i) 1, 2, 3, 4, 5, 6, 7, 8, or 9 fluorine atoms; and/or
(ii) 1, 2, or 3 substituents independently selected from the Group Two Substituents,
(3) phenyl, optionally substituted with 1, 2, or 3 substituents independently selected from Group Two Substituents; and
(4) 5- to 10-membered heteroaromatic, optionally substituted with 1, 2, or 3 substituents independently selected from the Group Two Substituents.

11. The conjugate molecule of claim 2, wherein the first and second linkers independently are selected from the group consisting of:

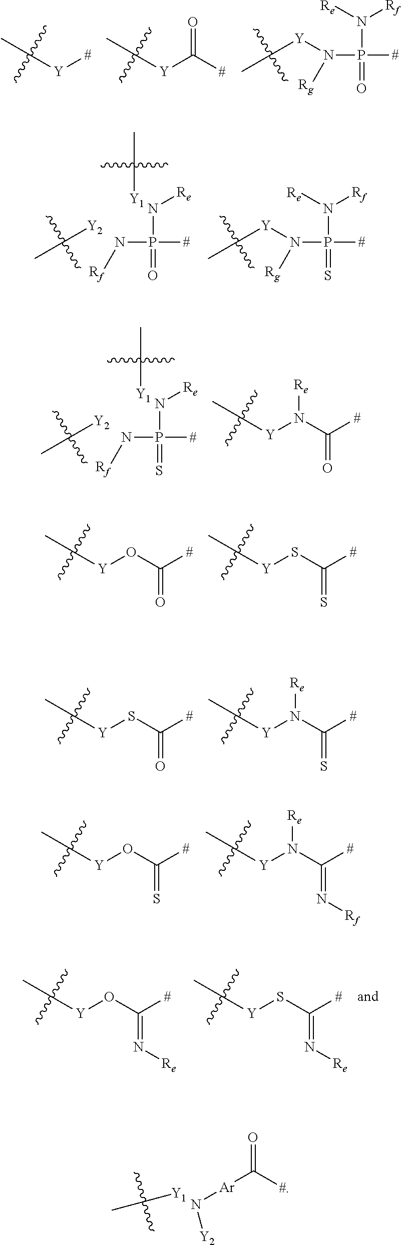

12. The conjugate molecule of claim 3, wherein the first, second, and third linkers independently are selected from the group consisting of:

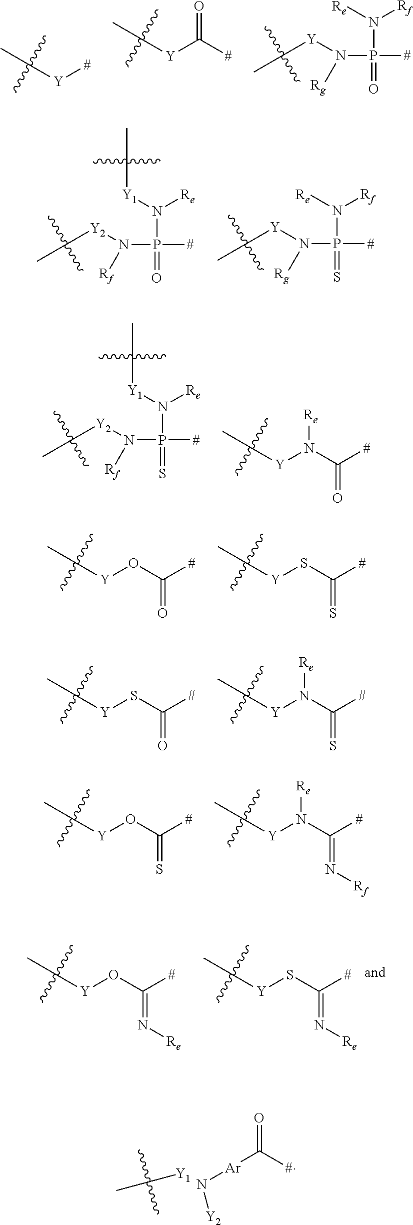

13. A pharmaceutically acceptable salt of the conjugate molecule of claim 1.

14. A composition comprising a conjugate molecule of claim 1, or a pharmaceutically acceptable salt thereof, and a pharmaceutically acceptable vehicle.

15. The composition of claim 14, which comprises;
(a) a racemic mixture of conjugate molecules or pharmaceutically acceptable salts thereof;
(b) a single enantiomer of the conjugate molecule or the pharmaceutically acceptable salt thereof;
(c) a mixture of diastereomers of the conjugate molecule or pharmaceutically acceptable salts thereof;
(d) a mixture of double bond isomers of the conjugate molecule or pharmaceutically acceptable salts thereof;
(e) a Z-double bond isomer of the conjugate molecule or the pharmaceutically acceptable salt thereof;
(f) a E-double bond isomer of the conjugate molecule or the pharmaceutically acceptable salt thereof; or
(g) an isotopic variant of the conjugate molecule or the pharmaceutically acceptable salt thereof.

16-21. (canceled)

22. A mixture comprising a plurality of conjugate molecules of claim 1, or pharmaceutically acceptable salts thereof, wherein the first avermectin component and the second avermectin component of each member of the plurality independently is an avermectin B₁ₐ component or an avermectin B_{1b} component.

23. The mixture of claim 22, which comprises a plurality of conjugate molecules comprising a first avermectin component, a first cannabinoid component, a second cannabinoid component, and, optionally, a third cannabinoid component.

24. The mixture of claim 23, wherein from about 1 to about 99% of the plurality comprises either the avermectin B₁ₐ component or the avermectin B_{1b} component.

25. The mixture of claim 23, wherein the ratio of conjugate molecules comprising the avermectin B₁ₐ component to conjugate molecules comprising the avermectin B_{1b} component is from about 75:25 to about 95:5.

26. A method of treating an infection caused by a virus, a parasite, or a helminth, comprising administering to an individual in need thereof a conjugate molecule of claim 1 or a pharmaceutically acceptable salt thereof.
